(19) United States
(12) Patent Application Publication
(10) Pub. No.: US 2025/0223195 A1
(43) Pub. Date: Jul. 10, 2025

(54) METHODS OF MAKING AND TESTING FUNCTIONAL WATER WITH ENHANCED CELL-PENETRATING CAPABILITY AND APPLICATIONS THEREOF

(71) Applicant: Golden Ally Lifetech Group Co., Ltd., Monrovia, CA (US)

(72) Inventor: YUNFENG LU, Culver City, CA (US)

(21) Appl. No.: 18/848,955

(22) PCT Filed: Jul. 25, 2022
(86) PCT No.: PCT/US2022/038130

§ 371 Date: Sep. 20, 2024

Related U.S. Application Data
(60) Provisional application No. 63/322,378, filed on Mar. 22, 2022.

(51) Int. Cl. C02F 1/00; A61K 33/00; A61P 39/06; C02F 1/36; G01N 33/50

(52) U.S. Cl. CPC C02F 1/005; A61K 33/00; A61P 39/06; C02F 1/36; G01N 33/5008

(57) ABSTRACT

A method of making functional water with enhanced cell-penetrating capability is provided. The method includes preparing a ceramic by melting natural clay and iron-containing sand, immerging the ceramic in water with a predetermined ceramic-to-water weight ratio at a predetermined temperature for a first predetermined period of time, and sonicating the ceramic in the water at predetermined sonication power at the predetermined temperature for a second predetermined period of time. The water being sonicated is then applied to flow through a column filled with the ceramic at the predetermined temperature to obtain the functional water. The water flows through the column with a predetermined water flow-to-ceramic weight ratio, or with a third predetermined time for a unit volume of the water passing through a unit weight of the ceramic. The ceramic induces structural change of at least some water molecules of the water.

### CROSS-REFERENCE TO RELATED PATENT APPLICATIONS

This PCT application claims priority to and the benefit of, pursuant to 35 U.S.C. § 119 (e), U.S. provisional patent application Ser. No. 63/322,378, filed Mar. 22, 2022, entitled “MAKING METHODS AND APPLICATIONS OF FUNCTIONAL WATER WITH ENHANCED CELL-PENETRATING CAPABILITY,” by Yungfeng Lu, which is incorporated herein in its entirety by reference.

### FIELD OF THE INVENTION

The present invention relates generally to water, and more particularly to methods of making and testing functional water with enhanced cell-penetrating capability and applications thereof.

### BACKGROUND OF THE INVENTION

The background description provided herein is for the purpose of generally presenting the context of the present invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions.

Water is a critical component for all organisms. Human beings are mostly water, which accounts for about 75 percent in infants, 50-60 percent in adult men and women, and as low as 45 percent of body mass in old age. From the uptake and process of nutrition to the removal of metabolic waste, all the chemical reactions and physiological processes take place in aqueous solutions. The average adult takes in about 2500 mL of aqueous fluids per day, mostly from the digestive tract, with 230 mL generated metabolically. About the same volume of water leaves the body per day, mostly as urine, through evaporation from the skin, and from air expelled from the lungs.

According to the Centers for Disease Control and Prevention (CDC), drinking water can prevent dehydration, a condition that can cause unclear thinking, result in mood change, cause your body to overheat, and lead to constipation and kidney stones. Besides water, there are many beverage options can be part of a healthy diet. Functional water, particularly, is water enhanced by supplementals such as vitamins, mineral, fruits, and herbs, which offers additional health and other functional benefits. Resulting from consumer preferences shifting from sugar and carbonate beverages to water, as well as the desires for more than only hydration, the past decades have witnessed a rapid market growth of functional water. Alkaline water, electrolyzed reduced water, and hydrogen-rich water, in particularly, have gained increasing interests due to their potential anti-inflammatory and antioxidant properties.

Therefore, a heretofore unaddressed need exists in the art to address the aforementioned deficiencies and inadequacies.

### SUMMARY OF THE INVENTION

In one aspect, the invention relates to a method of making aquaporin water with enhanced cell-penetrating capability. In one embodiment, the method includes: preparing a ceramic by melting natural clay and iron-containing sand; immerging the ceramic in water with a predetermined ceramic-to-water weight ratio at a predetermined temperature for a first predetermined period of time, and sonicating the ceramic in the water at predetermined sonication power at the predetermined temperature for a second predetermined period of time; and applying the water being sonicated to flow through a column filled with the ceramic at the predetermined temperature to obtain the aquaporin water, wherein the water flows through the column with a predetermined water flow-to-ceramic weight ratio, or with a third predetermined time for a unit volume of the water passing through a unit weight of the ceramic. The ceramic induces structural change of at least some water molecules of the water.

In one embodiment, water molecules of the aquaporin water have a H—O—H bond angle α, and a H—O—H bond angle α₁ of the changed water molecules is greater than a regular H—O—H bond angle α₀ of the water molecules of the water prior to being processed. In one embodiment, the H—O—H bond angle α₁ is about 120°, and the regular bond angle α₀ is 104.45°.

In one embodiment, the method further includes: verifying a plurality of capabilities of the aquaporin water by: preparing culture media by disposing growth enhancing materials in the aquaporin water, and filtering the aquaporin water to remove precipitations and bacteria in the aquaporin water; preparing control media by disposing the growth enhancing materials in deionized water; after preparing the culture media and the control media, disposing cells of a living subject respectively in the culture media and the control media; and measuring the cells in the culture media and the control media using a corresponding kit to determine the capabilities of the aquaporin water.

In one embodiment, the capabilities of the aquaporin water include: increasing viability of the cells; protecting the cells from oxidative injury; preserving telomere length of the cells; and downregulating genes of the living subject relevant to inflammation and aging.

In one embodiment, the ceramic is Tadanoumi ceramic, the predetermined ceramic-to-water weight ratio is 0.01-0.1 gram/ml of the ceramic to the water, the predetermined temperature is room temperature, the first predetermined period of time is 18-36 hours, the predetermined sonication power is 100 watt, the second predetermined period of time is 3-7.5 minutes, the predetermined water flow-to-ceramic weight ratio is 0.01 liter/minute/gram, and the third predetermined time is 0.01 second for 1 liter of the water passing through 1 gram of the ceramic.

In one embodiment, the growth enhancing materials include: a minimal essential medium (MEM) as a basal medium; and fetal bovine serum (FBS) and penecilin/streptomyosin (P/S) as supplemental materials.

Another aspect of the invention relates to aquaporin water prepared by the method as disclosed above. In certain embodiments, water molecules of the aquaporin water have a H—O—H bond angle α, and a H—O—H bond angle α₁ of the changed water molecules is greater than a regular H—O—H bond angle α₀ of the water molecules of the water prior to being processed. In one embodiment, the H—O—H bond angle di is about 120°, and the regular bond angle α₀ is 104.45°

The aquaporin water prepared by the method as disclosed above may be applied in various applications. For example, a further aspect relates to a method of enhancing removal of metabolic waste in a living subject, which includes: preparing the aquaporin water as discussed above, and providing the aquaporin water to the living subject. In certain embodiments, the aquaporin water is configured to enhance removal of the metabolic waste from cells of the living subject. In one embodiment, the metabolic waste includes hydrogen peroxide.

Yet a further aspect relates to a method, which includes: preparing the aquaporin water as discussed above, and providing the aquaporin water to the living subject. In certain embodiments, the aquaporin water may be configured to: increase the viability of the cells of the living subject, preserve the telomere length of the cells of the living subject, and/or be anti-inflammatory to downregulate the genes of the living subject.

In another aspect, the invention relates to a method of making functional water with enhanced cell-penetrating capability. In one embodiment, the method includes: preparing a ceramic by melting natural clay and iron-containing sand; immerging the ceramic in water with a predetermined ceramic-to-water weight ratio at a predetermined temperature for a first predetermined period of time to obtain the functional water, and sonicating the ceramic in the water at predetermined sonication power at the predetermined temperature for a second predetermined period of time; and applying the water being sonicated to flow through a column filled with the ceramic at the predetermined temperature to obtain the functional water, wherein the water flows through the column with a predetermined water flow-to-ceramic weight ratio, or with a third predetermined time for a unit volume of the water passing through a unit weight of the ceramic. The ceramic induces structural change of at least some of water molecules of the water.

In one embodiment, the method further includes verifying capabilities of the functional water by: disposing cells of a living subject in the functional water as culture media; disposing the cells of the living subject in in deionized water as control media; and measuring the cells in the culture media and the control media using a corresponding kit to determine the capabilities of the functional water. In one embodiment, the capabilities of the functional water include: increasing viability of the cells; protecting the cells from oxidative injury; preserving telomere length of the cells; and downregulating genes of the living subject relevant to inflammation and aging.

In one embodiment, the method further includes, prior to disposing the cells in the functional water: disposing growth enhancing materials in the culture media; and filtering the culture media to remove precipitations and bacteria in the functional water. In one embodiment, the growth enhancing materials include: a minimal essential medium (MEM) as a basal medium;

and fetal bovine serum (FBS) and penecilin/streptomyosin (P/S) as supplemental materials.

In one embodiment, the ceramic is Tadanoumi ceramic, and the functional water is aquaporin water. In one embodiment, the predetermined ceramic-to-water weight ratio is 0.01-0.1 gram/ml of the ceramic to the water. In one embodiment, the predetermined temperature is room temperature, the first predetermined period of time is 18-36 hours, the predetermined sonication power is 100 watt, the second predetermined period of time is 3-7.5 minutes, the predetermined water flow-to-ceramic weight ratio is 0.01 liter/minute/gram, and the third predetermined time is 0.01 second for 1 liter of the water passing through 1 gram of the ceramic.

Another aspect of the invention relates to aquaporin water prepared by the method as disclosed above. In certain embodiments, water molecules of the aquaporin water have a H—O—H bond angle α, and a H—O—H bond angle α₁ of the changed water molecules is greater than a regular H—O—H bond angle α₀ of the water molecules of the water prior to being processed. In one embodiment, the H—O—H bond angle α₁ is about 120°, and the regular bond angle α₀ is 104.45°.

Yet a further aspect of the invention relates to an aqueous solution, which includes the aquaporin water as disclosed above, where the changed water molecules of the aqueous solution have a volume V₁; unchanged water molecules of the aqueous solution have a volume Vo; the aqueous solution has a volume V, and V=V₁+V₀, and V₁/V₀>1.

The functional water prepared by the method as disclosed above may be applied in various applications. For example, a further aspect relates to a method of enhancing removal of metabolic waste in a living subject, which includes: preparing the functional water as discussed above, and providing the functional water to the living subject. In certain embodiments, the functional water may be configured to enhance removal of the metabolic waste from cells of the living subject. In one embodiment, the metabolic waste includes hydrogen peroxide.

Yet a further aspect relates to an aqueous solution, which includes: an amount of first water molecules in a first volume V₁, having a first average H—O—H bond angle di between two H—O bonds of each of the first water molecules; and an amount of second water molecules in a second volume V₀, having a second average H—O—H bond angle α₀ between two H—O bonds of each of the second water molecules.

In one embodiment, the first average H—O—H bond angle α₁ is greater than the second average H—O—H bond angle do. In one embodiment, the first average H—O—H bond angle α₁ is 120°, and the second average H—O—H bond angle α₀ is about 104.45°.

In one embodiment, the aqueous solution has a volume V, where V=V₁+V₀, and V₁/V₀>1.

These and other aspects of the present invention will become apparent from the following description of the preferred embodiments taken in conjunction with the following drawings, although variations and modifications thereof may be affected without departing from the spirit and scope of the novel concepts of the invention.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment.

FIG. 1 shows a flowchart of a method of making functional water with enhanced cell-penetrating capability according to certain embodiments of the invention.

FIG. 2 shows a flowchart of the process of verifying the capabilities of the functional water made by the method as shown in FIG. 1 according to certain embodiments of the invention.

FIG. 3 schematically shows geometry of an exemplary water molecule according to certain embodiments of the invention.

FIG. 4A schematically and qualitively shows, based on a proposed model, the potential relationship between the energy spectrum and the H—O—H bond angle of a water molecule according to certain embodiments of the invention.

FIG. 4B shows a proposed three-dimensional model indicating the volume and effective area of a water molecule related to the H—O—H bond angle according to certain embodiments of the invention.

FIG. 5 shows the features of AQP water with improved permeability in aquaporins, where (A) shows Relative values of AQP permeability (AQP pf) of AQP water made from distilled water (dw) vs. dw; (B) shows photographs of clay (top) and Tadanoumi ceramic (bottom); and (C) shows proliferation of human skin cells in control media and ceramic treated medium.

FIG. 6 shows (A) X-ray diffraction (XRD) pattern and (B) thermogravimetric analysis (TGA) of Tadanoumi ceramic according to certain embodiments of the invention.

FIG. 7 shows (A) relative cell viability of AML-12 cultured in DI-water and AQP-water., where P value: *<0.05, and (B) Relative cell death of HepG2 cultured in DI-water and AQP-water.

FIG. 8 shows (A) relative T/S ratios of the cells cultured in media made from DI water or AQP water as determined by qPCR, where P value: *<0.05; and (B) RTL ratios of the cells cultured in media made from DI water or AQP water as determined by qPCR.

FIG. 9 shows folds of gene expression changed in the cells cultured in AQP water over those in Control water as determined by RT-PCR.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this specification will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term are the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

It will be understood that, as used in the description herein and throughout the claims that follow, the meaning of “a”, “an”, and “the” includes plural reference unless the context clearly dictates otherwise. Also, it will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures. is turned over, elements described as being on the “lower” side of other elements would then be oriented on “upper” sides of the other elements. The exemplary term “lower”, can, therefore, encompasses both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising,” or “includes” and/or “including” or “has” and/or “having”, or “carry” and/or “carrying,” or “contain” and/or “containing,” or “involve” and/or “involving, and the like are to be open-ended, i.e., to mean including but not limited to. When used in this specification, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and this specification, and will not be interpreted in an idealized or overly formal sense unless expressly so defined herein.

As used in this specification, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in this specification, the phrase “at least one of A, B, and C” should be construed to mean a logical (A or B or C), using a non-exclusive logical OR. As used herein, the term “and/or” includes any and all combinations of one or more of the associated listed items.

The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in a different order (or concurrently) without altering the principles of the invention.

As discussed above, the past decades have witnessed a rapid market growth of functional water. One type of the functional water being made may be aquaporin (AQP) water.

Aquaporins (AQP): AQP water is a novel class of functional water that affords faster transport of water across cell membrane. Cells are the basic structural and functional units of life, comprising a cytoplasm enclosed with a cell membrane. Cell membrane provides a protective barrier around the cell while regulates which materials or molecule can pass in or not. Like ionic channels, such as calcium ion (Ca²⁺) channel, potassium ion (K⁺) channel, sodium ion (Na⁺) channel, and proton (H⁺) channel, that regulate the transport of ions across the cell membrane, the transport of water molecules, individually or in clusters, across the cell membrane is mediated through water channels, also called aquaporins, and driven by the osmosis pressure across the cell membrane. Aquaporins are membrane-intrinsic proteins and present in all organisms, which form pores in the cell membrane to facilitate the transport of water between cells. The existence of water channels has been speculated 60 years ago, and it was not until 1992 that the first aquaporin, aquaporin 1, was discovered by Peter Agre of John Hopkins University, who won the Nobel Prize in Chemistry in 2003 for his pioneering research in discovery of water channels.

Currently, in mammals, 13 aquaporins (AQPO-12) have been identified and are traditionally classified into three subgroups: 1) orthodox AQPs (AQPO, 1, 2, 5, 6, and 8) mainly for water transport, 2) aquaglyceroporins (AQP3, 7, 9, and 10), which in addition to water transport neutral solutes such as glycerol, urea, ammonia, and carbon dioxide, and 3) superaquaporins (AQP11 and 12), whose details are remained to be elucidated. In addition, hydrogen peroxide (H₂O₂) and water (H₂O) possess similar dipole moment and hydrogen-bonding capability. Like water, hydrogen peroxide transports across cell membranes also through aquaporins, including AQP3, 8, 9, and 11, a new subset of aquaporins termed peroxiporins. It is important to note that hydrogen peroxide is toxic; effective removal of hydrogen oxide from the body is essential for oxidative-stress reduction, anti-aging, prevention and treatment of many diseases.

AQP Water®. AQP water is a novel class of functional water that is made according to certain embodiments of the present invention. Compared with normal water or other functional water, AQP water has a unique function that allows water more effectively passthrough the aquaporins and cross the cell membranes, affording faster hydration and effective removal of toxic compounds such as hydrogen peroxide for human body.

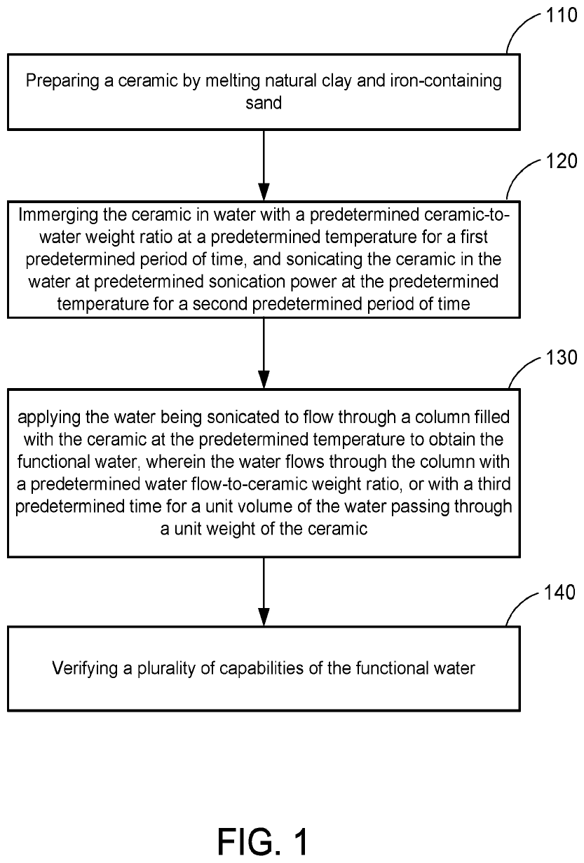

Certain aspects of the invention relate to methods of making functional water with enhanced cell-penetrating capability. For example, FIG. 1 shows a flowchart of a method of making functional water with enhanced cell-penetrating capability according to certain embodiments of the invention. It should be particularly noted that, unless otherwise stated in the present disclosure, the steps of the method may be arranged in a different sequential order, and are thus not limited to the sequential order as shown in FIG. 1.

As shown in FIG. 1, in the method of making functional water, at process 110, a ceramic is prepared by melting natural clay and iron-containing sand. In certain embodiments, the ceramic may also be Tadanoumi Ceramic®, which may be further discussed later. Then, at process 120, the ceramic is immerged in water with a predetermined ceramic-to-water weight ratio at a predetermined temperature for a predetermined period of time, and sonicating the ceramic in the water at predetermined sonication power at the predetermined temperature for a second predetermined period of time. During the process, the ceramic induces structural change of at least one or some water molecules of the water and reduces cluster size of the water. In certain embodiment, when the functional water is AQP water, the predetermined ceramic-to-water weight ratio may be in the range of 0.01-0.1 gram/ml of the ceramic to the water. In one embodiment, the predetermined ceramic-to-water weight ratio is in the range of 0.05-0.1 gram/ml of the ceramic to the water. In one embodiment, the predetermined temperature is room temperature, the first predetermined period of time is 18-36 hours, the predetermined sonication power is 100 watt, and the second predetermined period of time is 3.0-7.5 minutes. For example, the predetermined period of time may be 24 hours, and the second predetermined period of time may be 5 minutes.

Once the water is sonicated, at process 130, the sonicated water is applied to flow through a column filled with the ceramic (which is the same type of ceramic being immerged in the water) at the predetermined temperature to obtain the functional water. Specifically, the sonicated water flows through the column with a predetermined water flow-to-ceramic weight ratio, or with a third predetermined time for a unit volume of the sonicated water passing through a unit weight of the ceramic. In one embodiment, the predetermined water flow-to-ceramic weight ratio is 0.01 liter/minute/gram. In one embodiment, the third predetermined time is 0.01 second for 1 liter of the water passing through 1 gram of the ceramic.

Once the functional water is obtained, at process 140, the capabilities of the functional water may be verified. In one embodiment, the capabilities of the functional water that may be verified may include: increasing viability of the cells; protecting the cells from oxidative injury; preserving telomere length of the cells; and downregulating genes of the living subject relevant to inflammation and aging.

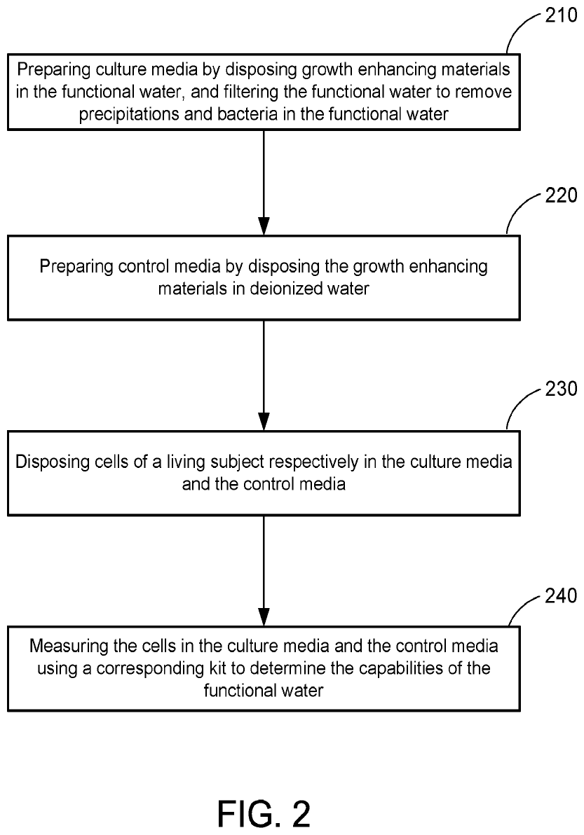

FIG. 2 shows a flowchart of the process of verifying the capabilities of the functional water made by the method as shown in FIG. 1 according to certain embodiments of the invention. It should be particularly noted that, unless otherwise stated in the present disclosure, the steps of the process may be arranged in a different sequential order, and are thus not limited to the sequential order as shown in FIG. 2.

As shown in FIG. 2, at process 210, culture media is prepared by disposing growth enhancing materials in the functional water, and filtering the functional water to remove precipitations and bacteria in the functional water. In one embodiment, the growth enhancing materials include: a minimal essential medium (MEM) as a basal medium; and fetal bovine serum (FBS) and penecilin/streptomyosin (P/S) as supplemental materials. At process 220, control media is prepared by disposing the growth enhancing materials in deionized water. After preparing the culture media and the control media, at process 230, cells of a living subject are disposed respectively in the culture media and the control media to perform the verification process. At process 240, the cells in the culture media and the control media are measured using a corresponding kit to determine the capabilities of the functional water.

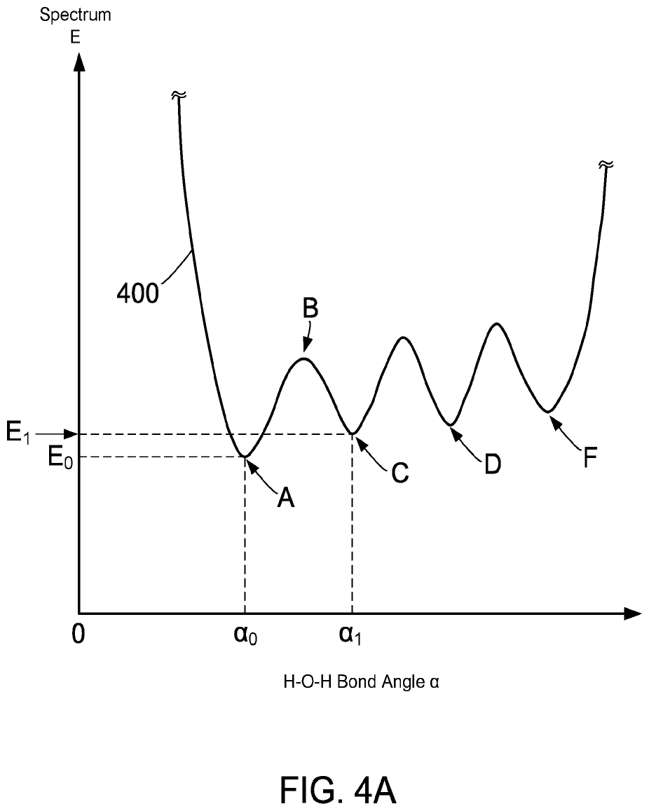

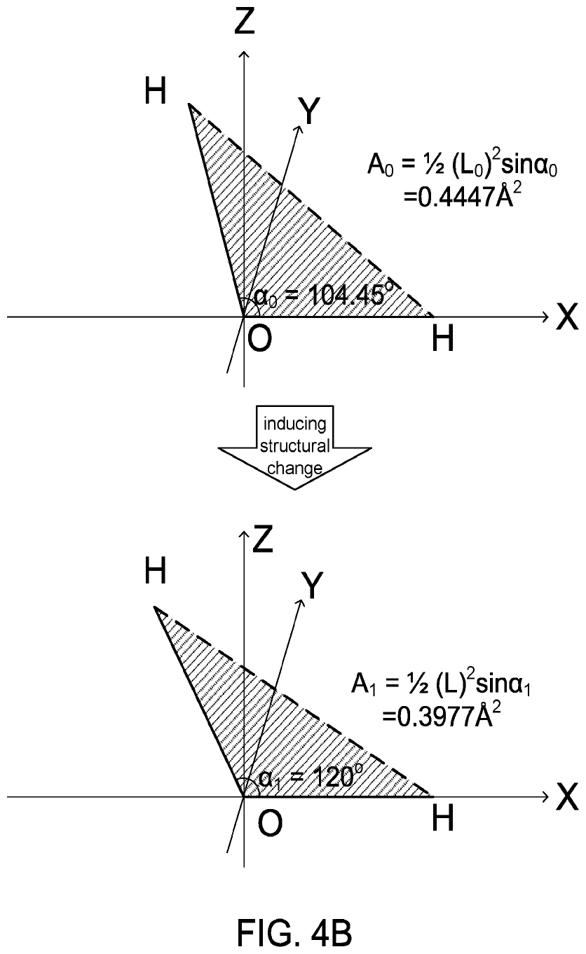

AQP water was first reported by Tadao Kozumi (Cosmic Com Ltd., Hiroshima, Japan) and Yoshichika Kitagawa (Akita Prefectural University, Japan), in “water structure changes induced by ceramics can be detected by increased permeability through aquaporin” (1), which was published in Biochemistry and Biophysics Reports (5, 2016, 353-358), and incorporated herein in its entirety by reference. Initially, AQP water was prepared simply by immerging Tadanoumi Ceramic® (see FIG. 4 (B)) in distilled water or tap water at room temperature for 24 h (4-gram ceramic in 50 mL water, which is equivalent to 0.08 gram/ml of the ceramic to the water). In comparison, in the method as shown in FIG. 1, in addition to the immerging process, the AQP water of the present invention is obtained by further sonicating the ceramic in the water after the immerging period, and the sonicated water is then further applied to flow through a column filled with the ceramic in order to filter the water. In this case, the structural change of molecules of the water induced by the Tadanoumi Ceramic and the reduced cluster size of the water may be more stabilized, thus enhancing the stability of the AQP water being obtained.

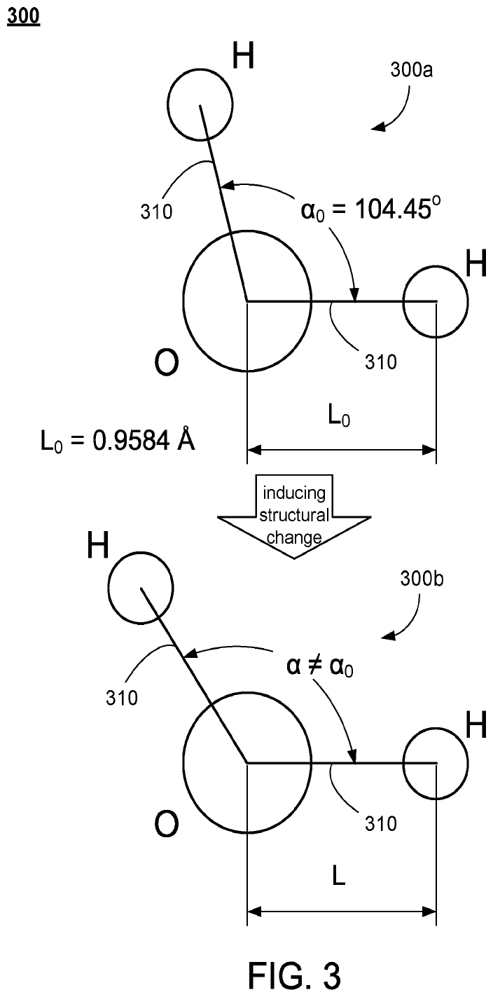

FIG. 3 shows schematically geometry of an exemplary water molecule according to certain embodiments of the invention. As discussed above, by performing the method of making functional water, there is an induced structural change of water molecules of the water. Specifically, as shown in FIG. 3, initially, the exemplary water molecule 300 at a regular state 300a has a H—O—H bond angle α₀ (which is an angle defined between the two H—O bonds 310) and a bond length L₀ (which is the length between the hydrogen and the oxygen along each H—O bond 310), and for regular water molecules, the regular bond angle α₀ on average is 104.45°, and the bond length L₀ is 0.9584 Å. However, for the functional water obtained through the process as described above according to certain embodiments of the present invention, at least one or some water molecules in state 300a are induced or changed to state 300b, where the H—O—H bond angle of the functional water molecules in state 300b is changed from ao to a, where a #ao, and the bond length L of the functional water molecules in state 300b may stay substantially unchanged or change correspondingly. In other words, the structure of the water molecule(s) 300 has been changed or modified during the process of making the functional water as set forth above.

In certain embodiments, once water molecules have been processed through the method of making functional water as described above, it has observed that the H—O—H bond angle α of the aquaporin water molecules on average is about a=α_{1=120.00}°. From such observation, a possible and plausible model regarding the H—O—H bond angle change was proposed (2). Specifically, FIG. 4A schematically and qualitively shows, based on the proposed model, the potential relationship between the energy spectrum and the H—O—H bond angle of a water molecule according to certain embodiments of the invention. As shown in FIG. 4A, where a proposed energy spectrum E of water molecules related to the H—O—H bond angle is schematically and qualitively shown, where point A is the first minimum of the energy spectrum curve 400. For regular water, water molecules stay in the energy ground state that is corresponding to point A on the energy spectrum curve 400, which may be characterized by energy E₀, and the corresponding regular bond angle α₀ is 104.45°. In making functional water according to certain embodiments of the present invention, the energy of the water molecules may be changed to cross the peak point B (which is a relative maximum point) and then reach the next relative minimum point C at the energy spectrum curve 400, where the corresponding H—O—H bond angle α₁ is about 120°, such that the changed or modified water molecules may be retained and remain stable for a certain period of time (e.g., more than one week). It should be noted that the energy spectrum curve 400 as shown may include multiple relative energy minimums, such that additional relative minimum points D and F, where the H—O—H bond angle α at each point respectively is further greater than the regular bond angle α₀ of 104.45°, may exist. In other words, in certain embodiments, with the water molecules being changed or modified, the average H—O—H bond angle α of the changed or modified water molecules may be greater than the regular H—O—H bond angle α₀ of 104.45° (e.g., points C, D and F).

It is observed that, with the changed or modified water molecules, the functional water may be easily consumable by human bodies and bring significant health benefits for human being, which may be further explained by the plausible model set forth above. (2) Specifically, FIG. 4B shows a proposed three-dimensional model indicating the volume and effective area of a water molecule related to the H—O—H bond angle according to certain embodiments of the invention. As partially shown in FIG. 4B, the water molecule is formed by the oxygen atom and the two hydrogen atoms, with both the H—O bonds on a same plane (hereinafter the X-Z plane on the three-dimensional coordinates), where the water molecule is formed in a three-dimensional space when the H—O—H bond area rotates in the X-Z plane and the Y-Z plane concurrently. Since the regular H—O—H bond angle α₀ of the regular water molecule is 104.45° and the H—O—H bond angle α₁ of the changed or modified water molecules is increased to be about 120°, the effective area (shown as the patterned area in FIG. 4B) will be reduced due to the increase of the bond angle (specifically, the effective area A₀ of the regular water molecules is about 0.4447 Å2, and the effective area A1 of the modifiled water molecules is about 0.3977 Å2), and based on the zero-order approximation, the effective volume and the corresponding surface S of the water molecule will be also correspondingly reduced. This results in a reduced surface tension of the changed or modified water molecules, which is shown as F=a. S. Thus, the hazardous free radicals generated by the human body may be more easily attached to and/or carried by the modified water molecules and removed from the body cells. On the other hand, the changed or modified water molecules of the functional water may provide enhanced permeation capabilities because of their smaller “effective volume.” The functional water made according to certain embodiemnts of the present invention thus contains a first amount of the changed or modified water molecules in a first volume V₁ and with a first H—O—H bond angle α₁, and a second amount of the regular water molecules in a second volume Vo and with a second H—O—H bond angle αo, where α₁>Δ₀. To be effective, the ratio of V₁/V₀ needs to be greater than one. In other words, V₁/V₀>1.

The unique capability of AQP water to more effectively pass through human aquaporins were demonstrated in oocytes that expressed human aquaporins AQP1, 2, 3, or 5. Briefly, oocytes in culture media were transferred in water. Water passed through the aquaporins swelling the cells driven by the osmotic pressure. The swelling rate of the cells was then measured and used to calculate the osmotic water permeability (Pf). It was found that, compared with tap water, Pf of the tap water treated with Tadanoumi Ceramic increased 26%, 48%, and 21% for the oocytes expressed AQP2, AQP3, and AQP5, respectively (statistical significance p<0.05). Consistently, compared with distilled water, Pf of distilled water treated with Tadanoumi Ceramic increased 2.2, 1.2, 1.3, and 1.6 folds for the oocytes expressed AQP1, AQP2, AQP3, and AQP5, respectively, as shown in FIG. 5 (A).

Meanwhile, it was found that APQ water also promoted the proliferation of normal human skin cells. As a demonstration, cell culture media was first placed with Tadanoumi Ceramic® (1 g ceramic in 50 mL medium, which is equivalent to 0.02 gram/ml of the ceramic to the water) at room temperature for 24 h. Full grown cells were trypsinized and used at a concentration of 1.0×10⁵cells/m. The cells cultured in control or ceramic-treated medium for 48 h were then treated with Cell counting kit-8. The number of viable cells was determined by measuring the absorbance at 450 nm if water soluble formazan, which was produced by intracellular dehydrogenase. As a result, the proliferation of the cells culture with the ceramic-treated medium was 12% higher than those in the control media (statistical significance p<0.05, see FIG. 5 (C)), suggesting that the use of ceramic-treated medium promoted the proliferation of cells.

Due to the capabilities of AQP water to more effectively pass through human aquaporins and to promote the proliferation of normal human skin cells, it is possible to apply the AQP water as a consumable aqueous solution. For example, an aqueous solution which contains at least 75% weight ratio of the AQP water as disclosed above may be used as a consumable solution for human beings.

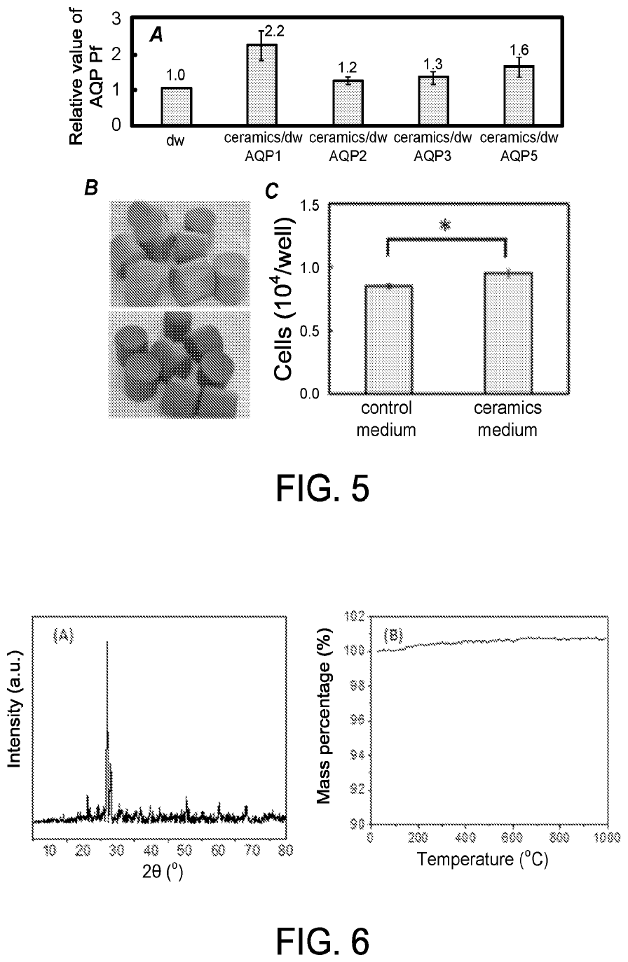

Tadanoumi Ceramic®: Tadanoumi Ceramic® is uniquely produced by melting natural clay and iron-containing sand from Hiroshima, Japan. FIG. 6 shows (A) X-ray diffraction (XRD) pattern and (B) thermogravimetric analysis (TGA) of Tadanoumi ceramic according to certain embodiments of the invention. As shown in FIG. 6 (A), the crystalline structure was examined using x-ray diffraction (XRD, Miniflex II, Rigaku, Cu Kα radiation 1.5406 Å, 40 kV, and current 30 mA, scan rate of 5° per minute). Tadanoumi ceramic shows a diffraction pattern that does not match any known pattern in the database of Joint Committee on Powder Diffraction Standards (JCPDS). Measured by nitrogen adsorption-desorption at 77 K, Tadanoumi ceramic is dense (non-porous) with a negligible surface area of 0.8 cm2/g. As shown in FIG. 6 (B), thermal stability was measured by thermogravimetric analysis (TGA, Netzsch STA 449 F3 Jupiter, an air flow rate 50 L/min, heating rate of 10° C./min). Tadanoumi ceramic showed negligible mass loss up to 1000° C., indicating a high thermal stability. In addition, Tadanoumi ceramic does not contain any detectable heavy metal as confirmed by x-ray fluorescence spectroscopic techniques.

The functional water prepared by the method as disclosed above may be applied in various applications. For example, a further aspect relates to a method of enhancing removal of metabolic waste in a living subject, which includes: preparing the functional water as discussed above, and providing the functional water to the living subject. In certain embodiments, the functional water may be configured to enhance removal of the metabolic waste from cells of the living subject, increase the viability of the cells of the living subject, preserve the telomere length of the cells of the living subject, and/or be anti-inflammatory to downregulate the genes of the living subject. In one embodiment, the metabolic waste includes hydrogen peroxide.

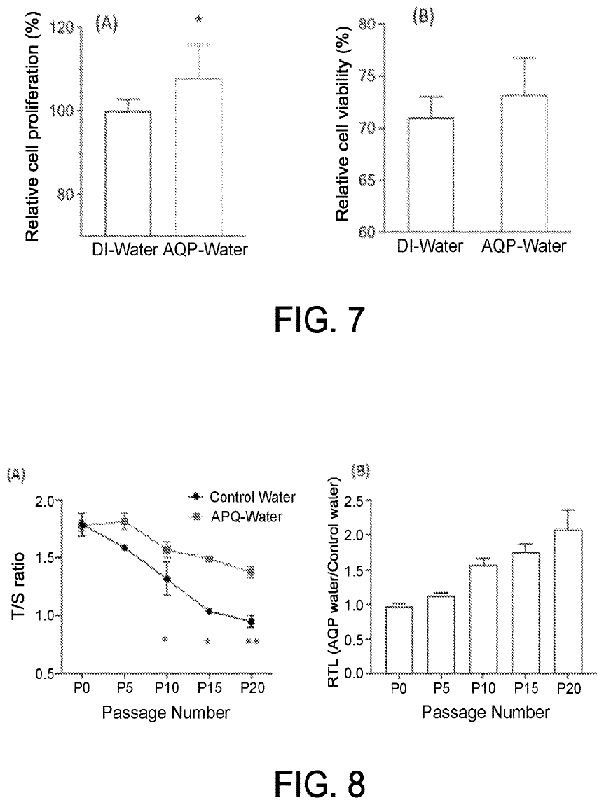

Ability in increasing cell viability and protecting cells from oxidative injury: The ability of AQP water in increasing cell viability was examined in AML-12 cells, a murine hepatocyte cell line derived from liver of transgenic mice. FIG. 7 shows (A) relative cell viability of AML-12 cultured in DI-water and AQP-water., where P value: *<0.05, and (B) Relative cell death of HepG2 cultured in DI-water and AQP-water. As shown in FIG. 7 (A), control culture media was prepared by dissolving Eagle's MEM (EMEM) powder (Sigma Aldrich) in deionization water (denoted as DI-Water) and supplemented with 10% FBS (Gibco) and 1% P/S (Genesee). Experimental culture media was prepared by dissolving EMEM powder (Sigma Aldrich) in AQP water (denoted as AQP-Water) and supplemented with 10% FBS (Gibco) and 1% P/S (Genesee). The culture media was filtered through a 0.22 μm PES vacuum filtration system (Genesee) to remove any precipitations and bacteria. AML-12 cells were seeded in a 96 well plate at 10,000 cells/well. The cells were cultured in media DI-Water and AQP-Water for 24 hours, respectively, and cell viability was measured with the Cell-titer blue kit (Sigma). The relative viability of cells cultured in AQP-Water was calculated using formula:

$$Relative cell viability ( \% ) = \frac{Cell viability with AQP - Water}{Cell viability with DI - Water} \times 100 \%$$

As shown in FIG. 7 (A), the cells showed cultured in AQP-Water showed a relative cell viability of 115% to those in DI-Water (statistical significance p<0.05), suggesting that AQP water increases the viability of cells.

The ability of AQP-Water to protect cells from oxidative injury was examined using HepG2, an immortal cell line derived from human hepatocellular carcinoma. As shown in FIG. 6 (B), control culture media was prepared by dissolving EMEM powder (Sigma Aldrich) in deionization water (denoted as DI-Water) and supplemented with 10% FBS (Gibco) and 1% P/S (Genesee). Experimental culture media was prepared by dissolving EMEM powder (Sigma Aldrich) in AQP active water (denoted as AQP-Water) and supplemented with 10% FBS (Gibco) and 1% P/S (Genesee). The Culture media was filtered through a 0.22 μm PES vacuum filtration system (Genesee) to remove any precipitations and bacteria. HepG2 cells were seeded in a 96 well plate at 10,000 cells/well. The Cells were first cultured in media DI-Water and AQP-Water for 24 hours, respectively. The Cells were then cultured for another 24 hours with a final concentration of 250 μM H₂O₂ added to the media. The cell viability was measured with the Cell-titer blue kit (Sigma). The relative cell viability of cells cultured in AQP-Water with H₂O₂ treatment was calculated using formula:

$$Relative cell viability ( \% ) = \frac{Cell viability before {H}_{2} {O}_{2} treatment}{Cell viability after {H}_{2} {O}_{2} treatment} \times 100 \%$$

The viability for cells cultured in AQP-Water was slightly higher than those in DI-Water (74% vs 71%).

Ability in preserving telomere length: Telomeres, the end parts of linear chromosomes, are nucleoprotein complexes consisting of highly conserved, tandem arrays of G-rich repetitive sequence. Telomeres preserve genome stability by preventing the ends of chromosomes from being recognized as broken DNA and triggering inappropriate responses associated with DNA damage. With cell division and/or oxidative stress, telomere length erodes until they reach a critically shortened length, at which point a permanent cell cycle is arrested and cells stop dividing. As an indicator of senescence and oxidative stress, telomere length has been recognized as a biomarker of aging and a risk factor for age-related diseases.

The ability of AQP water in preserving telomere length was examined with HEK-293, a human epithelial cell line derived from fetal kidney. The control culture media, denoted as Control water, was prepared by dissolving DMEM powder (Sigma Aldrich) in deionized water supplemented with 10% FBS (Gibco) and 1% of P/S (Genesee). The AQP culture media, denoted as AQP water, was prepared by dissolving DMEM powder (Sigma Aldrich) in AQP water supplemented with 10% FBS (Gibco) and 1% of P/S (Genesee). The culture media was filtered through a 0.22 μm PES vacuum filtration system (Genesee) to remove any precipitation and bacteria.

HEK-293 cells were cultured in T25 culture flasks in Control water and AQP water, respectively, which were sub-cultured every three days. The cellular genomic DNA from 5x105 cells sub-cultured for 5, 10, 15, and 20 passages, denoted as P5, P10, P15, and P20, respectively, was extracted using a DNA extraction kit (Qiagen). The telomere length was measured by a quantitative polymerase chain reaction (qPCR) method using a telomere primer pair of telg (5′-ACACTAAGGTTTGGGTTTGGGTTTGGGTTTGGGTTAGTGT-3′) and telc (5′-TGTTAGGTATCCCTATCCCTATCCCTATCCCTATCCCTAACA-3′). The number of the genomic gene was measured by qPCR using a beta-globin primer pair of Fwd 5′-GTGCGAGAGCGTCAGTATTAAG-3′and Rev 5′-TCCCTGCTTGCCCATACTA-3′.

FIG. 8 shows (A) relative T/S ratios of the cells cultured in media made from DI water or AQP water as determined by qPCR, where P value: *<0.05; and (B) RTL ratios of the cells cultured in media made from DI water or AQP water as determined by qPCR. Specifically, the qPCR testing was conducted using the following thermal cycling program consisting of Stage 1 (15 min at 95° C.), Stage 2 (2 cycles of 15 s at 94° C. and 15 s at 49° C.), and Stage 3 (32 cycles of 15 s at 94° C., 10 s at 62° C., and 15 s at 74° C. with signal acquisition). A standard curve of the Cq values vs. the DNA concentration was constructed for the telomere and genomic DNA, respectively, using MyiQ software (Bio-Rad iQ5 2.0 Standard Edition Optical System Software). The concentrations of telomere (T) and genomic DNA(S) in the samples were then calculated based on their Cq values, respectively. The T/S ratios were used to quantify the change of the telomere during cell growth. The RTL (relative telomere length) was also calculated by normalizing the concentration of telomere of the cells cultured in AQP water to those in Control water.

FIG. 8 (A) shows the T/S ratio of the cells cultured in control water or AQP water after 0, 5, 10, 15, and 20 passages. The cells showed consistently decreasing T/S ratio along with increasing passages, indicating a shortening telomere length during culture. Compared the cells in Control water, cells cultured in AQP water showed a significantly slow rate of T/S ratio decrease. FIG. 8 (B) shows the relative telomere length (RTL) of the cells cultured with AQP water vs. Control water. Consistent with the finding in FIG. 6 (B), RTL values increased with increasing passages. P5 showed RTL near 1.0, while P10, P15, and P20 showed significantly higher RTL of 1.56, 1.76, and 2.05 respectively, indicating AQP water can effective slowdown the shortening process of the telomere. Compared with Control water, AQP water increased approximately 56%, 76% and 105% of telomere length in P10, P15 and P20 cells, respectively. These results confirm the ability of AQP water to slowdown the shortening process of telomeres.

Ability in downregulating genes relevant to inflammation and aging: Nuclear factor-KB (NF-κB) plays a key role in inflammatory diseases. The activation of NF-κB induces the expression of various pro-inflammatory genes, regulating the production of proinflammatory cytokines, recruitment of leukocytes, and formation of inflammasomes. Deregulated NF-κB activation is a hallmark of chronic inflammatory diseases, such as diabetes, cancer, and neurodegenerative diseases. For many chronic diseases, oxidative stress is another critical risk factor. The Keap1-Nrf2 [Kelch-like ECH-associated protein 1-nuclear factor (erythroid-derived 2)-like 2] regulatory pathway is the principal inducible defense against oxidative stress, where Nrf2 mediates antioxidant response, and its inhibitor Keap1 acts as a sensor for oxidative stress.

The ability of AQP water in downregulating genes relevant to inflammation and aging was examined in HEK-293, a human epithelial cell line derived from fetal kidney. The control culture media, denoted as Control water, was prepared by dissolving DMEM powder (Sigma Aldrich) in deionized water supplemented with 10% FBS (Gibco) and 1% of P/S (Genesee). The AQP culture media, denoted as AQP water, was prepared by dissolving DMEM powder (Sigma Aldrich) in AQP active water supplemented with 10% FBS (Gibco) and 1% of P/S (Genesee). The culture media was filtered through a 0.22 μm PES vacuum filtration system (Genesee) to remove any precipitation and bacteria.

HEK-293 cells were cultured in T25 culture flasks in Control water and AQP water, respectively, which sub-cultured every three days for 27 passages. The cellular RNA from 5x105 cells was extracted using a RNA extraction kit (Qiagen). The isolated cellular RNA was converted into cDNA with cDNA reverse transcription kits (Qiagen) through a reverse transcription polymerase chain reaction (RT-PCR) method using random primers. The representative genes relevant to inflammation and aging were monitored by polymerase chain reaction (RT-PCR) method using primer pairs of NF-κB (Fwd 5′-GCACCCTGACCTTGCCTATTT-3′ and Rev 5′-GTCCCAGGCGCCTTGTGAAGC-3′), Nrf2 (Fwd 5′-GAGACAGGTGAATTTCTCCCAAT-3′ and Rev 5′-TTTGGGAATGTGGGCAAC-3′) and Keap1 (Fwd 5′-GGAGGACCACACCAAGCAAGC-3′ and Rev 5′-GGATGAAGCCAGCACCACCTTG-3′). The GAPDH gene was measured as internal control using primers of Fwd 5′-GCACCGTCAAGGCTGAGAAC-3′and Rev 5′-TGGTGAAGACGCCAGTGGA-3′.

The PCR testing was conducted using the thermal cycling program consisting of Stage 1 (15 min at 95° C.), Stage 2 (2 cycles of 15 s at 94° C. and 15 s at 49° C.), and Stage 3 (32 cycles of 15 s at 94° C., 10 s at 62° C., and 15 s at 74° C. with signal acquisition). The gene expression levels of GAPDH (glyceraldehyde-3-phosphate dehydrogenase), NF-κB, Nrf2, and Keap1 were calculated with MyiQ software (Bio-Rad iQ5 2.0 Standard Edition Optical System Software). The DNA amounts of cells cultured in AQP water and Control water were calculated based on their Cq values and normalized with GAPDH expression.

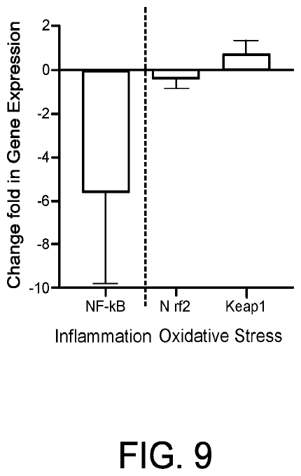

FIG. 9 shows folds of gene expression changed in the cells cultured in AQP water over those in Control water as determined by RT-PCR. Specifically, FIG. 9 shows the change fold of gene expression in cells cultured for 27 passages in AQP water than those in Control water. Compared to the cells cultured in Control water, AQP water suppressed NF-κB gene expression for 5 folds, indicating an anti-inflammation effect. In addition, cells cultured in AQP water showed 0.5-fold decrease in Nrf2 expression. Constantly, AQP water enhanced 0.7-fold expression of Keap1, the inhibitor of Nrf2, in comparison with Control water. This result suggests that AQP water can help to release oxidative stress during cell aging.

Potential functions and benefits of AQP water: The studies presented above suggest that AQP water passes through aquaporins faster than normal water and exhibits antioxidative and anti-inflammatory capability, as well as the ability to preserve telomeres. The action mechanism of AQP water remains elusive and requires further studies. Kozumi and Kitagawa proposed that the enhanced permeation of AQP water could be attributed to Tadanoumi ceramic, which induced structural change of at least one or some water molecules and reduced the size of water clusters. Based on the roles of aquaporins that enable the transport of water and metabolites across the cell members, as-observed functions of the AQP water could be attributed to the faster permeation of AQP water that leads to more effective removal of metabolic waste such as hydrogen peroxide.

The AQP water prepared by the method as disclosed above may be applied in various applications. For example, a further aspect relates to a method of enhancing removal of metabolic waste in a living subject, which includes: preparing the AQP water as discussed above, and providing the AQP water to the living subject. In certain embodiments, the AQP water is configured to enhance removal of the metabolic waste from cells of the living subject. In one embodiment, the metabolic waste includes hydrogen peroxide.

Hydrogen peroxide is the major content of reactive oxygen species (ROS), which are mainly generated as byproducts of cellular metabolism through the electron transport chains in mitochondria and cytochrome P450. The other major source is enzymatic reactions mediated by oxidases (e.g., NAPDH oxidase), which are ubiquitously present in a variety of cells, particularly phagocytes and endothelial cells. In human being and other organisms, hydrogen peroxide is mainly eliminated by catalase and broke down into water and oxygen. Catalase is the most abundant antioxidant enzyme ubiquitously present in the liver, erythrocytes, and alveolar epithelial cells.

Hydrogen peroxide not only serves as a weapon against pathogens (e.g., bacteria) due to its cytotoxicity, it also serves as a signaling molecule for many physiological processes. Unbalanced production and elimination of hydrogen peroxide results in local or systematic accumulation of hydrogen peroxide, causing oxidative stress and cell apoptosis through DNA damage, lipid peroxidation and protein oxidation. Extended research has linked ROS to diseases such as cancer, insulin resistance, diabetes mellitus cardiovascular diseases, atherosclerosis, aging, gout, and chronical inflammations. Enabling effective transport of hydrogen peroxide across the cell membranes, as well as their subsequent breakdown into water and oxygen, is critical to reduce the oxidative stress and maintain a healthy homeostasis. In this context, the inventors believe that AQP water is a truly unique class of functional water that may bring significant benefits for human being health.

The functional water as discussed above may be applied in various applications. For example, in a further aspect, an aqueous solution may be made using the functional water. In certain embodiments, for example, the aqueous solution may includes: an amount of first water molecules (i.e., the functional water with the changed or modified water molecules) in a first volume V₁, having a first average H—O—H bond angle α₁ (i.e., about) 120° between two H—O bonds of each of the first water molecules; and an amount of second water molecules (i.e., the regular water molecules) in a second volume V₀, having a second average H—O—H bond angle αo (e.g.,) 104.45° between two H—O bonds of each of the second water molecules. In one embodiment, the first average H—O—H bond angle α₁ (e.g.,) 120° is greater than the second average H—O—H bond angle α₀(i.e.,) 104.45°. In one embodiment, the aqueous solution has a volume V, where V=V₁+V₀, and V₁/V₀>1.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

Some references, which may include patents, patent applications, and various publications, are cited and discussed in the description of this invention. The citation and/or discussion of such references is provided merely to clarify the description of the invention and is not an admission that any such reference is “prior art” to the invention described herein. All references cited and discussed in this specification are incorporated herein by reference in their entireties and to the same extent as if each reference was individually incorporated by reference.

### REFERENCE LIST

1. Kozumi T, Kitagawa Y., “Water structure changes induced by ceramics can be detected by increased permeability through aquaporin,” Biochem. and Biophys. Rep. (2016 Jan. 8, 5:353-358), doi: 10.1016/j.bbrep.2016.01.002.
2. Private communications with Dr. Tim Tingkang Xia, May 30-Jun. 1, 2022.

1. A method of making functional water or aquaporin water with enhanced cell-penetrating capability, comprising:
preparing a ceramic by melting natural clay and iron-containing sand;
immerging the ceramic in water with a predetermined ceramic-to-water weight ratio at a predetermined temperature for a first predetermined period of time, and sonicating the ceramic in the water at predetermined sonication power at the predetermined temperature for a second predetermined period of time; and
applying the water being sonicated to flow through a column filled with the ceramic at the predetermined temperature to obtain the aquaporin water, wherein the water flows through the column with a predetermined water flow-to-ceramic weight ratio, or with a third predetermined time for a unit volume of the water passing through a unit weight of the ceramic;
wherein the ceramic induces structural change of at least some water molecules of the water.

2. The method of claim 1, wherein water molecules of the aquaporin water have a H—O—H bond angle α, and a H—O—H bond angle di of the changed water molecules is greater than a regular H—O—H bond angle α₀ of the water molecules of the water prior to being processed.

3. The method of claim 2, wherein the H—O—H bond angle α₁ is about 120°, and the regular bond angle α₀ is 104.45°.

4. The method of claim 1, further comprising:
verifying a plurality of capabilities of the aquaporin water by:
preparing culture media by disposing growth enhancing materials in the aquaporin water, and filtering the aquaporin water to remove precipitations and bacteria in the AQP water;
preparing control media by disposing the growth enhancing materials in deionized water;
after preparing the culture media and the control media, disposing cells of a living subject respectively in the culture media and the control media; and
measuring the cells in the culture media and the control media using a corresponding kit to determine the capabilities of the aquaporin water.

5. The method of claim 4, wherein the capabilities of the functional water or aquaporin water include:
increasing viability of the cells;
protecting the cells from oxidative injury;
preserving telomere length of the cells; and
downregulating genes of the living subject relevant to inflammation and aging.

6. The method of claim 1, wherein the ceramic is Tadanoumi ceramic, the predetermined ceramic-to-water weight ratio is 0.01-0.1 gram/ml of the ceramic to the water, the predetermined temperature is room temperature, the first predetermined period of time is 18-36 hours, the predetermined sonication power is 100 watt, the second predetermined period of time is 3-7.5 minutes, the predetermined water flow-to-ceramic weight ratio is 0.01 liter/minute/gram, and the third predetermined time is 0.01 second for 1 liter of the water passing through 1 gram of the ceramic.

7. The method of claim 1, wherein the growth enhancing materials include:
a minimal essential medium (MEM) as a basal medium; and
fetal bovine serum (FBS) and penecilin/streptomyosin (P/S) as supplemental materials.

8. Aquaporin water prepared by the method of claim 1, wherein water molecules of the aquaporin water have a H—O—H bond angle α, and a H—O—H bond angle α₁ of the changed water molecules is greater than a regular H—O—H bond angle α₀ of the water molecules of the water prior to being processed.

9. The aquaporin water of claim 8, wherein the H—O—H bond angle α₁ is about 120°, and the regular bond angle α₀ is 104.45°.

10. (canceled)

11. A method of enhancing removal of metabolic waste in a living subject, comprising:
preparing the aquaporin water of claim 1; and
providing the aquaporin water to the living subject, wherein the aquaporin water is configured to enhance removal of the metabolic waste from cells of the living subject.

12. The method of claim 11, wherein the metabolic waste includes hydrogen peroxide.

13. A method of increasing viability of cells of a living subject, comprising:
preparing the aquaporin water of claim 1; and
providing the aquaporin water to the living subject, wherein the aquaporin water is configured to increase the viability of the cells of the living subject.

14. A method of protecting cells of a living subject from oxidative injury, comprising:
preparing the aquaporin water of claim 1; and
providing the aquaporin water to the living subject, wherein the aquaporin water is configured to be antioxidative to protect the cells of the living subject from oxidative injury.

15. A method of preserving telomere length of cells of a living subject, comprising:
preparing the aquaporin water of claim 1; and
providing the aquaporin water to the living subject, wherein the aquaporin water is configured to preserve the telomere length of the cells of the living subject.

16. A method of downregulating genes of a living subject relevant to inflammation and aging, comprising:
preparing the aquaporin water of claim 1; and
providing the aquaporin water to the living subject, wherein the aquaporin water is configured to be anti-inflammatory to downregulate the genes of the living subject.

17. (canceled)

18. The method of claim 17, further comprising verifying capabilities of the functional water by:
disposing cells of a living subject in the functional water as culture media;
disposing the cells of the living subject in in deionized water as control media; and
measuring the cells in the culture media and the control media using a corresponding kit to determine the capabilities of the functional water.

19. (canceled)

20. The method of claim 18, further comprising, prior to disposing the cells in the functional water:
disposing growth enhancing materials in the functional water as the culture media; and
filtering the culture media to remove precipitations and bacteria in the functional water.

21-27. (canceled)

28. An aqueous solution, comprising:
an amount of first water molecules in a first volume V₁, having a first average H—O—H bond angle α₁ between two H—O bonds of each of the first water molecules; and
an amount of second water molecules in a second volume V₀, having a second average H—O—H bond angle α₀ between two H—O bonds of each of the second water molecules,
wherein the aqueous solution has a volume V, and V=V₁+V₀, and V₁/V₀>1.

29. The aqueous solution of claim 28, wherein the first average H—O—H bond angle α₁ is greater than the second average H—O—H bond angle do.

30. The aqueous solution of claim 29, wherein the first average H—O—H bond angle α₁ is 120°, and the second average H—O—H bond angle α₀ is about 104.45°.

31. (canceled)
